(19) United States
(12) Patent Application Publication
(10) Pub. No.: US 2026/0090705 A1
(43) Pub. Date: Apr. 2, 2026

(54) OPERATION PART FOR MEDICAL DEVICE, ULTRASOUND PROBE, AND ENDOSCOPE

(71) Applicant: FUJIFILM Corporation, Tokyo (JP)

(72) Inventors: Takuro IWAKI, Kanagawa (JP); Shintaro HATTORI, Kanagawa (JP); Koichi ISHIDA, Kanagawa (JP)

(73) Assignee: FUJIFILM Corporation, Tokyo (JP)

(21) Appl. No.: 19/344,547

(22) Filed: Sep. 30, 2025

(30) Foreign Application Priority Data
Sep. 30, 2024 (JP) 2024-170804

(51) Int. Cl. A61B 1/005; A61B 90/00

(52) U.S. Cl. CPC A61B 1/0052; A61B 90/08; A61B 2090/0807

(57) ABSTRACT

An operation part for a medical device includes: an exterior member; an operation lever provided in an operation part main body, which is a first region of the exterior member, rotatably about a rotation shaft in a state of being erected in a Z(+) direction; one arc surface that is provided in a grip part, which is a second region on a base end part side of the operation part main body, and that has a normal component on a Z(-) side; another arc surface that is provided in the operation part main body, has a normal component on the Z(-) side, and that has a distance to a longitudinal axis equal to or less than a distance from the arc surface to the longitudinal axis; and a locking part provided on a distal end part side of the rotation shaft of the operation lever and on the Z(-) side.

### CROSS-REFERENCE TO RELATED APPLICATION

The present application claims priority under 35 U.S.C §119(a) to Japanese Patent Application No. 2024-170804 filed on September 30, 2024, which is hereby expressly incorporated by reference, in its entirety, into the present application.

### BACKGROUND OF THE INVENTION

### 1. Field of the Invention

The present invention relates to an operation part for a medical device, an ultrasound probe, and an endoscope.

### 2. Description of the Related Art

In an endoscope, which is one type of medical device, a bendable part of an insertion part is bent by operating an operation lever provided in an endoscope operation part. Accordingly, a distal end part of the insertion part can be oriented in a desired direction.

JP2007-089966A discloses an endoscope comprising a main body operation part having an angle operation lever and a hard insertion part having an angle part. An operator grips the main body operation part and operates the angle operation lever to bend the angle part.

The main body operation part of JP2007-089966A has a main body casing to which the angle operation lever is attached. A front casing and a rear casing are connected to both ends of the main body casing, the insertion part extends from the front casing, and a universal cord is pulled out from the rear casing. In addition, the main body casing is configured as a large-diameter portion having a larger outer diameter (diameter) than that of the front casing and the rear casing.

### SUMMARY OF THE INVENTION

In the main body operation part (operation part for a medical device) disclosed in JP2007-089966A, the angle operation lever (operation lever) is rotationally operated with a thumb in a state where an index finger is hooked on the main body casing, but, in this case, the index finger may slip on the main body casing, or the main body casing may get in the way and cause the thumb to come off the operation lever. That is, the operation part for a medical device of JP2007-089966A has a problem in that it is difficult for an operator to grip the operation part and operate the operation lever.

The present invention has been made in view of such circumstances, and an object of the present invention is to provide an operation part for a medical device, which is easy to grip and has an operation lever that is easy to operate, an ultrasound probe, and an endoscope.

In order to achieve the above-described object, according to a first aspect of the present invention, there is provided an operation part for a medical device, the operation part comprising: an exterior member that has a distal end part and a base end part and that has a longitudinal axis defined by the distal end part and the base end part; at least one operation lever that is provided in a first region, which is a partial region, between the distal end part and the base end part of the exterior member and that is configured to be rotatable about a rotation shaft in a state of being erected on one side in a first direction orthogonal to the longitudinal axis; a first outer peripheral surface that is provided in a second region of the exterior member on a base end part side with respect to the first region and that has a normal component directed toward the other side opposite to the one side in the first direction; a second outer peripheral surface that is provided in the first region, that has a normal component directed toward the other side in the first direction, and that has a distance to the longitudinal axis equal to or less than a distance from the first outer peripheral surface to the longitudinal axis; and an indicator part that is provided on a distal end part side with respect to the rotation shaft and on the other side in the first direction.

According to a second aspect of the present invention, in the operation part for a medical device according to the first aspect, an end part of the indicator part on the base end part side is located on the distal end part side with respect to the rotation shaft.

According to a third aspect of the present invention, the operation part for a medical device according to the first or second aspect further comprises: a third outer peripheral surface that is provided in the second region, that is a surface intersecting a second direction orthogonal to each of the longitudinal axis and the first direction, and that has a distance to the longitudinal axis shorter than the distance from the first outer peripheral surface to the longitudinal axis.

According to a fourth aspect of the present invention, in the operation part for a medical device according to the third aspect, an end part of the third outer peripheral surface on the distal end part side is located on the base end part side with respect to the rotation shaft.

According to a fifth aspect of the present invention, in the operation part for a medical device according to the third or fourth aspect, the third outer peripheral surface is a flat surface.

According to a sixth aspect of the present invention, in the operation part for a medical device according to any one of the first to fifth aspects, the at least one operation lever includes a first operation lever that bends an insertion part connected to the distal end part in a first bending direction, and a second operation lever that bends the insertion part in a second bending direction different from the first bending direction, and a rotation shaft of the first operation lever is formed to extend in a direction in which a rotation shaft of the second operation lever extends.

According to a seventh aspect of the present invention, in the operation part for a medical device according to any one of the first to sixth aspects, the indicator part has a locking surface configured to be hooked by a finger, and a distance from the locking surface to the longitudinal axis is shorter than the distance from the first outer peripheral surface to the longitudinal axis.

According to an eighth aspect of the present invention, in the operation part for a medical device according to any one of the first to seventh aspects, the indicator part has a friction coefficient greater than a friction coefficient of the first outer peripheral surface.

According to a ninth aspect of the present invention, there is provided an ultrasound probe comprising: the operation part for a medical device according to any one of the first to eighth aspects; and an insertion part connected to the distal end part of the operation part for a medical device, in which a probe is provided on a distal end side of the insertion part.

According to a tenth aspect of the present invention, there is provided an endoscope comprising: the operation part for a medical device according to any one of the first to eighth aspects; and an insertion part connected to the distal end part of the operation part for a medical device, in which an observation window is provided on a distal end side of the insertion part.

According to the present invention, the operation lever is easy to grip and easy to operate.

### BRIEF DESCRIPTION OF THE DRAWINGS

FIG. 1 is an overall perspective view of an ultrasound probe according to an embodiment.

FIG. 2 is a left side view of the ultrasound probe shown in FIG. 1.

FIG. 3 is a right side view of the ultrasound probe shown in FIG. 1.

FIG. 4 is a top view of the ultrasound probe shown in FIG. 1.

FIG. 5 is a bottom view of the ultrasound probe shown in FIG. 1.

FIG. 6 is a perspective view of a main part of an operation part main body of an operation part as viewed from below.

FIG. 7 is an explanatory view showing an example of a case where an operator operates an operation lever.

FIG. 8 is an explanatory view showing an outer shape of a grip part in a case where the grip part is viewed in a direction of a longitudinal axis.

FIG. 9 is a left side view of the operation part in a case where the operation part is viewed from a left side.

FIG. 10 is an explanatory view showing a state in which the operator inserts an insertion part into a body cavity.

### DESCRIPTION OF THE PREFERRED EMBODIMENTS

Hereinafter, an operation part for a medical device, an ultrasound probe, and an endoscope according to an embodiment of the present invention will be described with reference to the accompanying drawings.

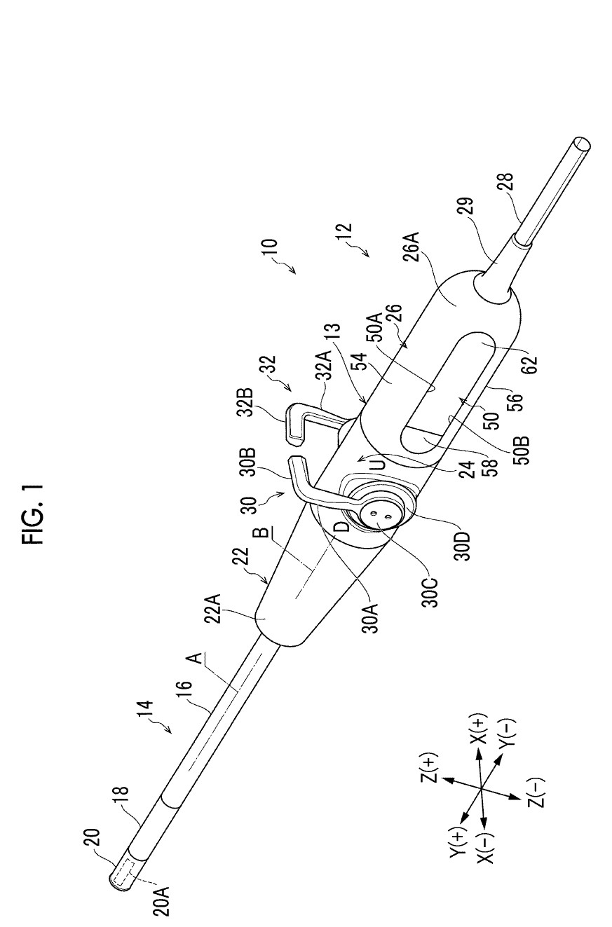

FIG. 1 is an overall perspective view of an ultrasound probe 10 according to an embodiment to which the operation part for a medical device of the embodiment of the present invention is applied. Hereinafter, in describing a configuration of each part of the ultrasound probe 10, an XYZ three-dimensional rectangular coordinate system will be used for the sake of convenience of description.

That is, a Z direction in the drawing refers to an up-down direction, a Z(+) direction refers to an up direction, and a Z(-) direction refers to a down direction. The Z direction corresponds to a first direction of the present invention, and a Z(+) direction side and a Z(-) direction side correspond to one side and the other side opposite to the one side in the first direction of the present invention. In addition, an X direction in the drawing refers to a left-right direction perpendicular to the Z direction, an X(+) direction refers to a right direction, and an X(-) direction refers to a left direction. The X direction corresponds to a second direction of the present invention. In addition, a Y direction in the drawing refers to a direction perpendicular to both the Z direction and the X direction, a Y(+) direction refers to a distal end direction, and a Y(-) direction refers to a base end direction. In the present specification, the expression “orthogonal” includes an angle between two directions with a slight variation with respect to 90° (for example, 90°±10°).

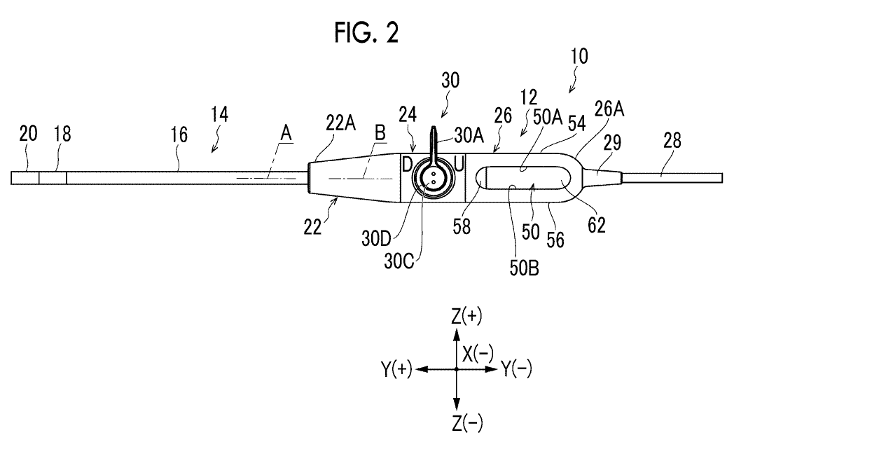

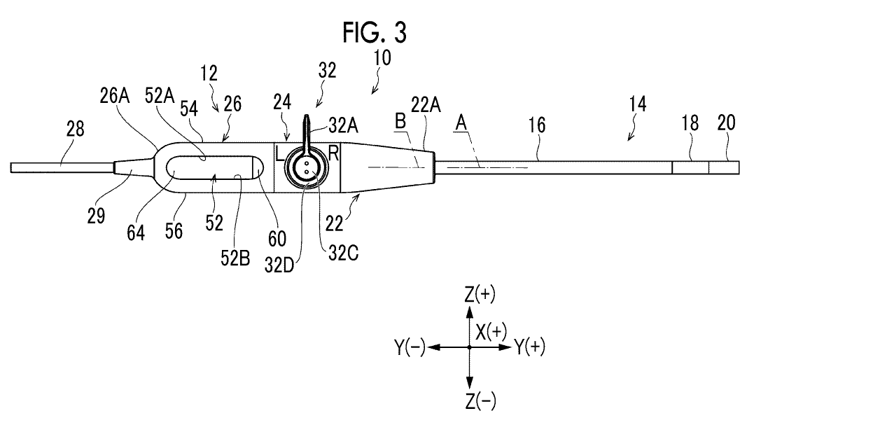

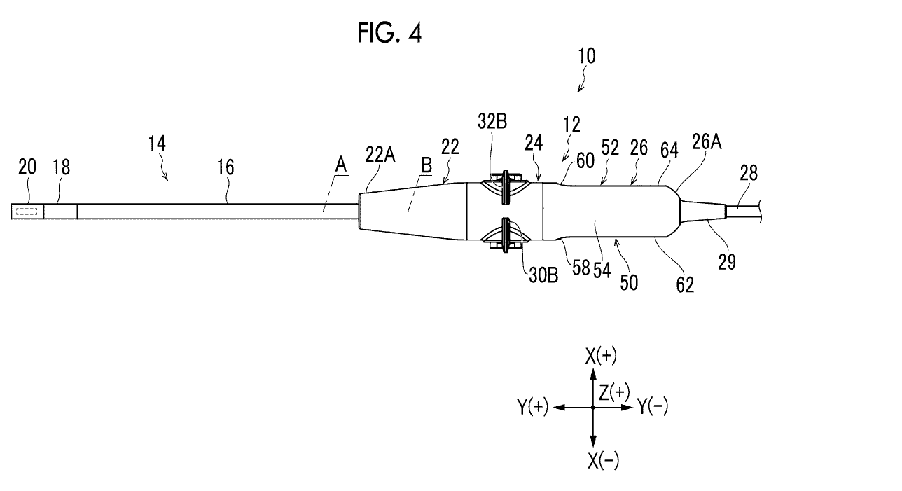

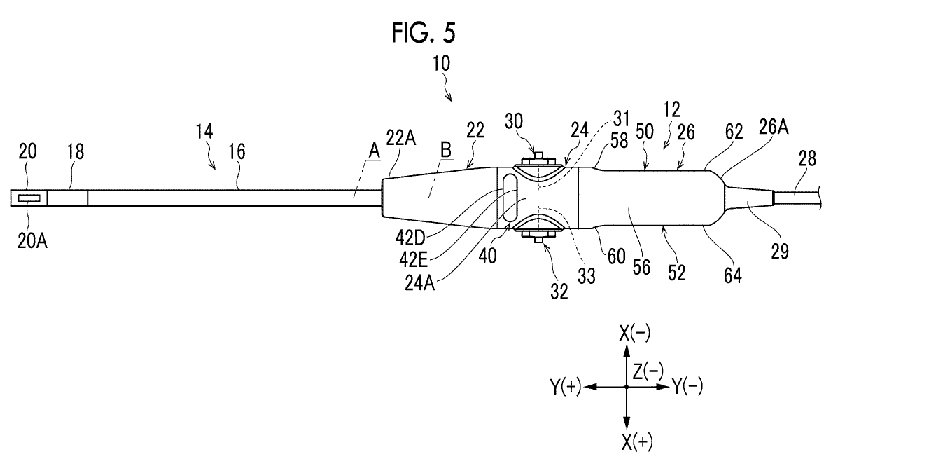

FIGS. 2 and 3 are left and right side views of the ultrasound probe 10 shown in FIG. 1 as viewed from a left side (X(-) side) and a right side (X(+) side), respectively. In addition, FIGS. 4 and 5 are top and bottom views of the ultrasound probe 10 shown in FIG. 1 as viewed from an upper side (Z(+) side) and a lower side (Z(-) side), respectively.

As shown in FIGS. 1 to 5, the ultrasound probe 10 comprises an operation part 12, an insertion part 14, and a cable 28. The operation part 12 is an example of an operation part for a medical device of the embodiment of the present invention. In addition, the insertion part 14 is an example of an insertion part connected to a distal end part of the present invention.

The insertion part 14 is configured in a tubular shape, and has a longitudinal axis A defined by its distal end and base end. In addition, the insertion part 14 includes a rigid insertion part main body 16, a bendable part 18 provided on a distal end side (Y(+) side) of the insertion part main body 16, and a probe 20 provided on a distal end side of the bendable part 18.

The bendable part 18 is configured to be bendable, for example, by consecutively disposing a plurality of ring-shaped bendable pieces (not shown) along the longitudinal axis A. The bendable part 18 is bent in the up-down direction (Z direction) and the left-right direction (X direction) by rotational operation of an operation lever 30 and an operation lever 32 described below.

The probe 20 is a linear type probe having a flat ultrasonic wave emission surface 20A, and has a plurality of transducers that transmit and receive ultrasonic waves to and from an imaging part (an organ, for example, a liver) of a subject. The probe 20 is connected to an external ultrasound imaging device (not shown) via a wiring line (not shown). The probe 20 is not limited to a linear type probe, and may be a convex type or radial type probe.

Next, a configuration of the operation part 12 will be described. As shown in FIG. 1, the operation part 12 comprises an exterior member 13.

### Exterior Member

The exterior member 13 has a distal end part 22A and a base end part 26A, and has a longitudinal axis B defined by the distal end part 22A and the base end part 26A. The distal end part 22A, the base end part 26A, and the longitudinal axis B are examples of a distal end part, a base end part, and a longitudinal axis of the present invention, respectively. The longitudinal axis B and the longitudinal axis A are axes along the Y direction.

The operation lever 30 and the operation lever 32 are provided in a partial region (first region) between the distal end part 22A and the base end part 26A of the exterior member 13. As shown in FIG. 5, the operation lever 30 and the operation lever 32 are configured to be rotatable about a rotation shaft 31 and a rotation shaft 33, respectively. The operation lever 30 and the operation lever 32 are examples of at least one operation lever of the present invention, and are examples of a first operation lever and a second operation lever of the present invention.

The rotation shaft 31 of the operation lever 30 is formed to extend in a direction in which the rotation shaft 33 of the operation lever 32 extends. In the operation part 12 of this example, the rotation shaft 31 and the rotation shaft 33 are formed along the X direction orthogonal to the longitudinal axis B, and the rotation shaft 31 and the rotation shaft 33 are formed on the same axis line. Although the rotation shaft 31 of this example is formed on the same axis line as the rotation shaft 33, the rotation shaft 31 need only extend in the direction in which the rotation shaft 33 extends, and may be formed to extend, for example, in the X direction parallel (including substantially parallel) to the X direction in which the rotation shaft 33 extends. The rotation shafts 31 and 33 are examples of rotation shafts of the present invention.

The exterior member 13 comprises an operation part-distal end part 22 having the distal end part 22A and a grip part 26 having the base end part 26A. In addition, the exterior member 13 comprises an operation part main body 24 provided between the operation part-distal end part 22 and the grip part 26. The operation lever 30 and the operation lever 32 are rotatably provided on the operation part main body 24. The operation part main body 24 and the grip part 26 are examples of a first region and a second region of the present invention, respectively.

As described above, the exterior member 13 is configured such that the operation part-distal end part 22, the operation part main body 24, and the grip part 26 are consecutively disposed from the distal end side (Y(+) side) to a base end side (Y(-) side) of the longitudinal axis B. In addition, the operation part-distal end part 22, the operation part main body 24, and the grip part 26 are each configured in a tubular shape with the longitudinal axis B as a central axis.

### Operation Part-Distal End Part

The operation part-distal end part 22 is configured in a truncated cone shape in which an outer diameter gradually decreases toward the distal end part 22A. A base end part of the insertion part 14 is connected to the distal end part 22A.

### Operation Part Main Body

The operation part main body 24 is configured in a cylindrical shape. As described above, the operation lever 30 and the operation lever 32 for bending the bendable part 18 are rotatably provided on an outer peripheral surface of the operation part main body 24. Hereinafter, the operation levers 30 and 32 will be described.

### Operation Lever

The operation lever 30 and the operation lever 32 are operation members for bending the bendable part 18 in the up-down direction and in the left-right direction. The operation lever 30 and the operation lever 32 are provided on a left side surface and a right side surface of the outer peripheral surface of the operation part main body 24, respectively, the left side surface and the right side surface facing each other with the longitudinal axis B interposed therebetween. That is, the operation lever 30 and the operation lever 32 are disposed at positions shifted from each other by 180 degrees in a circumferential direction of the operation part main body 24.

In addition, each position of the operation lever 30 and the operation lever 32 shown in FIGS. 1 to 5 indicates a neutral position (non-operation position) before the bendable part 18 is bent. In the neutral position, the operation lever 30 and the operation lever 32 are in a state of being erected in the up direction (Z(+) direction: one side in the first direction). In this state, the operation lever 30 and the operation lever 32 are configured to be rotatable about the rotation shaft 31 and the rotation shaft 33.

In addition, the operation lever 30 and the operation lever 32 are configured in an L-shape that is line-symmetrical with respect to the longitudinal axis B. That is, the operation lever 30 and the operation lever 32 have an arm part 30A and an arm part 32A that extend upward (Z(+) side) from the outer peripheral surface of the operation part main body 24, respectively.

In addition, the operation lever 30 has a finger rest part 30B that extends from an upper end of the arm part 30A to the right side (X(+) side). In addition, the operation lever 32 has a finger rest part 32B that extends from an upper end of the arm part 32A to the left side (X(-) side).

The operation lever 30 having an L-shape is configured by the arm part 30A and the finger rest part 30B, and the operation lever 32 having an L-shape is configured by the arm part 32A and the finger rest part 32B. An operator rotates the operation lever 30 or the operation lever 32 while placing a pad of his or her thumb on the finger rest part 30B or the finger rest part 32B.

For example, as shown in FIG. 2, in a case where the operation lever 30 in the erected state is rotated in a clockwise direction (U direction) or in a counterclockwise direction (D direction) about the rotation shaft 31, the bendable part 18 is bent upward or downward. In addition, as shown in FIG. 3, in a case where the operation lever 32 in the erected state is rotated in a clockwise direction (R direction) or in a counterclockwise direction (L direction) about the rotation shaft 33, the bendable part 18 is bent to the right or left. By rotating the operation lever 30 or the operation lever 32 in this way, the bendable part 18 is bent in the up-down direction, which is a first bending direction, or in the left-right direction, which is a second bending direction.

In addition, disk-shaped rotating portions 30C and 32C are provided at lower ends of the arm part 30A and the arm part 32A, respectively. The rotating portion 30C and the rotating portion 32C are rotatably supported by a circular protruding portion 30D and a circular protruding portion 32D provided in the operation part main body 24. The circular protruding portions 30D and 32D have a diameter larger than diameters of the rotating portions 30C and 32C, and the rotating portions 30C and 32C are stably supported by the circular protruding portions 30D and 32D.

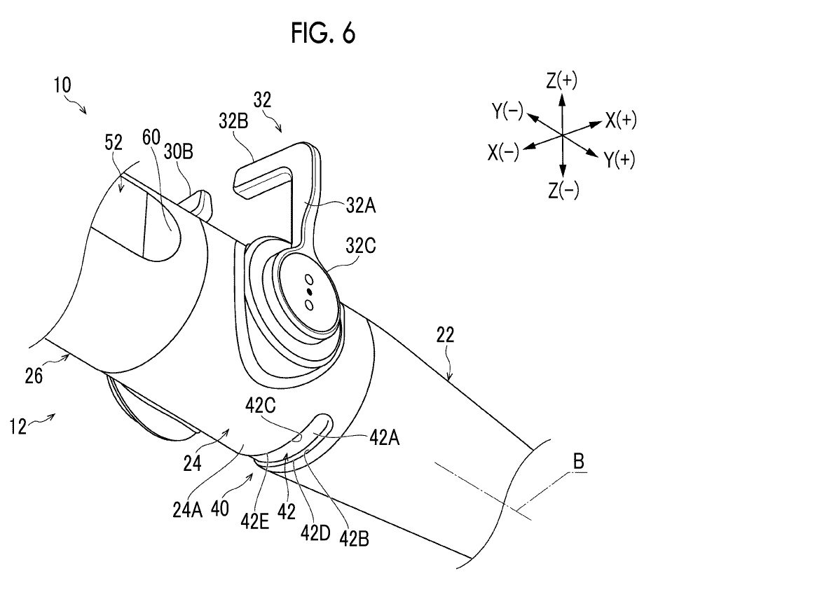

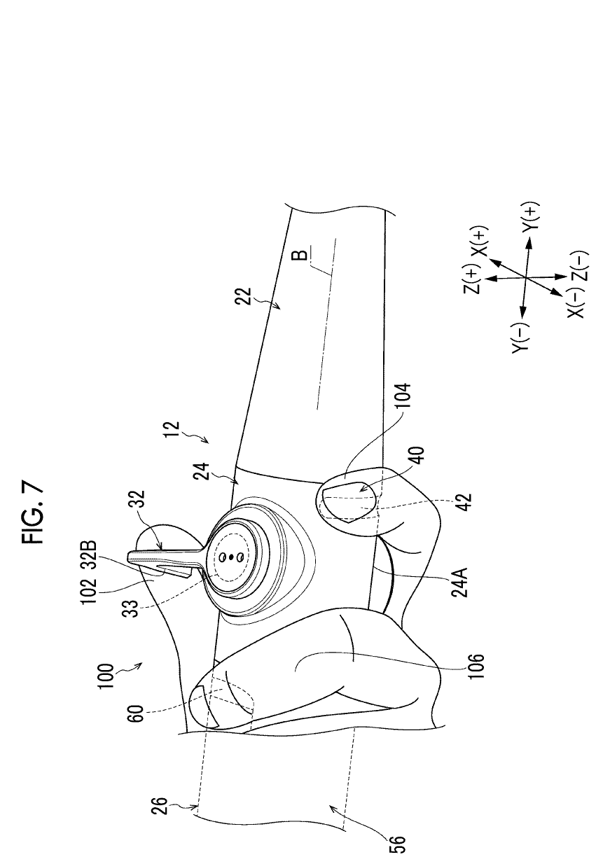

FIG. 6 is a perspective view of a main part of the operation part main body 24 of the operation part 12 as viewed from below (Z(-) side). FIG. 7 is an explanatory view showing an example of a case where the operator operates the operation lever 32.

### Locking Part

As shown in FIGS. 6 and 7, the operation part main body 24 comprises a locking part 40. The locking part 40 is a portion that serves as an indicator for a finger (index finger) of the operator to be placed on, and is an example of an indicator part of the present invention.

The locking part 40 is provided on a surface on the distal end part 22A side with respect to the rotation shafts 31 and 33 of the operation levers 30 and 32 shown in FIG. 5 and on an arc surface 24A on the opposite side (Z(-) side) to an erecting direction (Z(+) direction) of the operation levers 30 and 32.

In addition, the locking part 40 is configured as a recessed groove part 42. The recessed groove part 42 is provided along a circumferential direction about the longitudinal axis B. The recessed groove part 42 has a bottom surface 42A, and a distal end-side wall surface 42B and a base end-side wall surface 42C that connect the bottom surface 42A and the arc surface 24A. With the locking part 40 having such a configuration, in a case where a fingertip of the index finger is placed on the locking part 40, a pad of the fingertip is hooked on the bottom surface 42A. The bottom surface 42A is an example of a locking surface configured to be hooked by a finger of the present invention.

Further, in the locking part 40, a distal end edge part 42D and a base end edge part 42E are formed between the distal end-side wall surface 42B and the arc surface 24A and between the base end-side wall surface 42C and the arc surface 24A, respectively. The distal end edge part 42D and the base end edge part 42E are formed along the circumferential direction. In addition, the base end edge part 42E is located on the distal end part 22A side with respect to the rotation shafts 31 and 33 of the operation levers 30 and 32. The base end edge part 42E is an example of an end part of the indicator part on the base end part side of the present invention.

Furthermore, the locking part 40 is formed in a groove shape in which a depth of the bottom surface 42A increases from both end parts of the bottom surface 42A toward the center in the circumferential direction such that the entire pad of the fingertip is hooked on the bottom surface 42A. The depth of the bottom surface 42A in the circumferential direction may be uniform.

As will be described in detail below, as shown in FIG. 7, in a case where the operator grips the operation part 12 with a left hand 100 and rotates the operation lever 32 (which may be the operation lever 30; the same applies below) with a thumb 102 of the left hand 100, the operator rotates the operation lever 32 while hooking a fingertip of an index finger 104 of the left hand 100 on the locking part 40 (recessed groove part 42).

### Grip Part

Returning to FIG. 1, the grip part 26 is configured in a cylindrical shape having the same outer diameter as an outer diameter of the operation part main body 24. In addition, the grip part 26 has a base end part 26A configured in a hemispherical shape, and a distal end part of the cable 28 is connected to the base end part 26A via a bending prevention part 29.

The grip part 26 is a portion gripped by a palm of the operator's left hand or right hand. While gripping the grip part 26, the operator inserts and removes the insertion part 14 into and from a body of the subject, and rotates the operation lever 30 or the operation lever 32. Hereinafter, the grip part 26 will be described in detail.

As shown in FIGS. 1 to 5, the grip part 26 comprises two holding surfaces 50 and 52, and two arc surfaces 54 and 56 on an outer peripheral surface of the grip part 26.

The holding surface 50 and the holding surface 52 are formed in a substantially rectangular shape having a long side along the longitudinal axis B in plan view as seen from the left-right direction (X direction). In addition, the holding surface 50 and the holding surface 52 are provided on a left side surface and a right side surface that face each other with the longitudinal axis B interposed therebetween. That is, the holding surface 50 and the holding surface 52 are formed at positions shifted from each other by 180 degrees in the circumferential direction of the grip part 26.

The arc surface 54 and the arc surface 56 are arc surfaces that connect the holding surface 50 and the holding surface 52 in the circumferential direction of the grip part 26 about the longitudinal axis B. In addition, the arc surface 54 and the arc surface 56 are provided on an upper surface and a lower surface that face each other with the longitudinal axis B interposed therebetween. That is, the arc surface 54 and the arc surface 56 are disposed at positions shifted from each other by 180 degrees in the circumferential direction of the grip part 26.

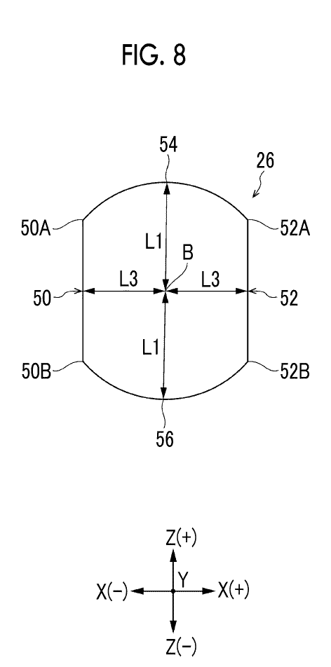

FIG. 8 is an explanatory view showing an outer shape of the grip part 26 in a case where the grip part 26 is viewed in a direction of the longitudinal axis B. As shown in FIG. 8, the holding surface 50 and the holding surface 52 are surfaces orthogonal to the X direction, which is the second direction, and a distance L3 to the longitudinal axis B is shorter than a distance L1 from the arc surface 54 and the arc surface 56 to the longitudinal axis B. In addition, the holding surface 50 and the holding surface 52 are each formed as a flat surface. Although the holding surfaces 50 and 52 of this example are surfaces orthogonal to the X direction, the holding surfaces 50 and 52 need only be surfaces intersecting the X direction, and may be, for example, surfaces inclined with respect to the surfaces orthogonal to the X direction. In addition, the holding surfaces 50 and 52 of this example are flat surfaces, but since these are portions that come into contact with the palm as will be described below, the holding surfaces 50 and 52 may be slightly distorted. The holding surfaces 50 and 52 are each an example of a third outer peripheral surface of the present invention.

With the grip part 26 having such a configuration, edge parts 50A and 52A are formed at respective connecting portions between the holding surfaces 50 and 52 and the arc surface 54, and edge parts 50B and 52B are formed at respective connecting portions between the holding surfaces 50 and 52 and the arc surface 56.

In addition, as shown in FIGS. 4 and 5, inclined surfaces 58 and 60 that are inclined from the holding surfaces 50 and 52 toward the outer peripheral surface of the grip part 26 are formed at end parts of the holding surfaces 50 and 52 on the distal end part 22A side, respectively. The inclined surfaces 58 and 60 are located on the base end part 26A side with respect to the rotation shafts 31 and 33. Each of the end parts on which the inclined surfaces 58 and 60 are formed is an example of an end part side of the third outer peripheral surface on the distal end part side of the present invention.

In addition, at end parts of the holding surfaces 50 and 52 on the base end part 26A side, D-cut flat surfaces 62 and 64 are formed by cutting off a part of the hemispherical outer peripheral surface of the base end part 26A along the longitudinal axis B. The above is the configuration of the grip part 26. Since the flat surfaces 62 and 64 are also the portions that come into contact with the palm, these may be slightly distorted.

Incidentally, in the field of medical equipment such as an ultrasound probe and an endoscope, there is a demand for an operation part for a medical device that is easy for the operator to grip and whose operation lever is easy to operate in order to smoothly perform a medical treatment. Therefore, the operation part 12 of the embodiment has the following configuration in order to provide such an operation part for a medical device.

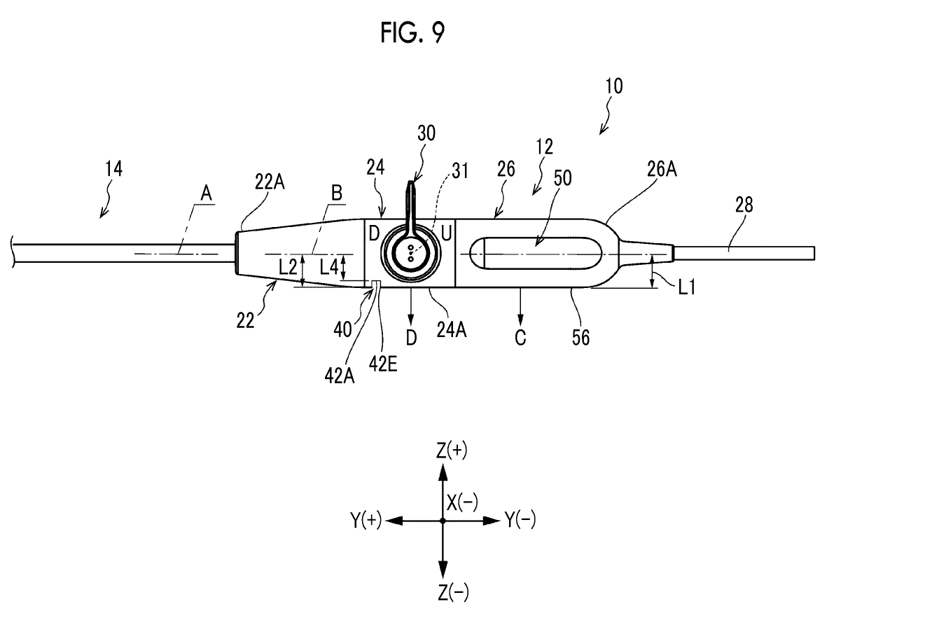

FIG. 9 is a left side view of the operation part 12 in a case where the operation part 12 is viewed from the left side (X(-) side). FIG. 9 shows only necessary reference numerals in comparison with the left side view shown in FIG. 2.

As shown in FIG. 9, the operation part 12 includes the operation part main body 24 as a first region where the operation lever 30 is provided, and the grip part 26 as a second region provided on the base end part 26A side with respect to the operation part main body 24.

In addition, the operation part 12 has the arc surface 56 in the grip part 26, which is the second region, and the arc surface 56 has a normal component C directed toward the lower side (Z(-) side). A distance from the arc surface 56 to the longitudinal axis B is a distance L1. The arc surface 56 is an example of a first outer peripheral surface of the present invention. In this example, the arc surface 56 is exemplified as the first outer peripheral surface, but the first outer peripheral surface is not limited to the arc surface, and may be an outer peripheral surface formed of a curved surface or a flat surface.

In addition, the operation part 12 has the arc surface 24A in the operation part main body 24, which is the first region, and the arc surface 24A has a normal component D directed toward the lower side (Z(-) side). A distance L2 from the arc surface 24A of this example to the longitudinal axis B is equal to the distance L1 (L2 = L1), and the arc surface 24A is formed to be flush with the arc surface 56. In addition, the distance L2 may be less than the distance L1 (L2 < L1). In this case, the arc surface 24A is formed at a position on the upper side (Z(+) side) with respect to the arc surface 56. The arc surface 24A is an example of a second outer peripheral surface of the present invention. In this example, the arc surface 24A is exemplified as the second outer peripheral surface, but the first outer peripheral surface is not limited to the arc surface, and may be an outer peripheral surface formed of a curved surface or a flat surface.

In addition, the operation part 12 includes the locking part 40. The locking part 40 is provided on the distal end part 22A side with respect to the rotation shaft 31 of the operation lever 30 and on the lower side (Z(-) side). In addition, the base end edge part 42E, which is an end part of the locking part 40 on the base end part 26A side, is located on the distal end part 22A side with respect to the rotation shaft 31 of the operation lever 30. In addition, the bottom surface 42A, which is the locking surface, has a distance L4 from the longitudinal axis B that is shorter than the distance L1 from the arc surface 56 to the longitudinal axis B.

Next, the operation part 12 of the embodiment will be compared with the main body operation part of JP2007-089966A.

The main body operation part (hereinafter, referred to as a “comparative example operation part”) of JP2007-089966A has an operation lever that is provided to be erected on the main body casing, in which an outer peripheral surface of the main body casing on a side opposite to an erecting direction side of the operation lever protrudes in an arc shape.

In a case where the comparative example operation part is described in comparison with the operation part 12 of the embodiment, the operation lever of the comparative example operation part is provided to be rotatable in a state of being erected on the upper side (Z(+) side) of the main body casing, and a lower surface of the main body casing is formed as a protruding surface that protrudes in an arc shape toward the lower side (Z(-) side). The operator rotates the operation lever with the thumb in a state where the index finger is placed on the protruding surface, but, in this case, there is a problem in that the index finger slips on the protruding surface, or the protruding surface gets in the way and causes the thumb to come off the operation lever.

With respect to such a comparative example operation part, the operation part 12 of the embodiment does not comprise a protruding surface as in the comparative example operation part, since the arc surface 24A of the operation part main body 24 and the arc surface 56 of the grip part 26 are in a relationship of L2 ≤ L1. That is, the arc surface 24A on which the index finger is placed is formed to be flush with the arc surface 56 held in the palm or is formed at a position on the upper side (Z(+) side) with respect to the arc surface 56.

In a case where the operator rotates the operation lever 30 with the thumb 102 in a state where the index finger 104 is placed on the arc surface 24A, the above-described configuration (L2 ≤ L1) prevents the index finger 104 from slipping on the arc surface 24A and allows the operator to rotate the operation lever 30 while preventing the thumb 102 from coming off the operation lever 30. As a result, the operation part 12 of the embodiment can solve the above-described problem of the comparative example operation part.

In addition to the above-described configuration, the operation part 12 of the embodiment comprises the locking part 40 provided on the distal end part 22A side with respect to the rotation shaft 31 of the operation lever 30 and on the lower side (Z(-) side). In addition, the base end edge part 42E, which is an end part of the locking part 40 on the base end part 26A side, is located on the distal end part 22A side with respect to the rotation shaft 31.

In a case where the index finger 104 is hooked on the locking part 40, the palm of the thumb 102 faces the finger rest part 30B of the operation lever 30 in the Z direction, so that the thumb 102 can be guided to an optimum position with respect to the operation lever 30. In addition, in this case, the palm can be guided to a good position with respect to the grip part 26. That is, by providing the locking part 40 at the above-described position, the entire hand of the operator can be guided to a good position with respect to the operation part 12.

Therefore, with the operation part 12 of the embodiment, the arc surface 24A (first outer peripheral surface) of the operation part main body 24 is configured not to protrude downward (Z(-) side) further than the arc surface 56 (second outer peripheral surface) of the grip part 26, and the locking part 40 (indicator part) that is a portion serving as an indicator for a finger (index finger) of the operator to be placed on is provided on the arc surface 24A of the operation part main body 24 (more specifically, at a position on the distal end part 22A side with respect to the rotation shafts 31 and 33 of the operation levers 30 and 32), making it easier to grip the operation part 12 and easier to operate the operation lever 30.

In addition, with the operation part 12 of the embodiment, as shown in FIG. 7, in a case where the operator grips the grip part 26 with the left hand 100 and the thumb 102 is placed on the finger rest part 32B of the operation lever 32, the palm (not shown) of the left hand 100 is placed on the holding surface 50 (see FIG. 1), and fingertips of a middle finger 106, a ring finger (not shown), and a little finger (not shown) are placed on the holding surface 52 (see FIG. 3). Therefore, the operation part 12 can be reliably held with the left hand 100.

In this case, the palm is caught on the edge parts 50A and 50B of the holding surface 50 and respective joints of the middle finger 106, the ring finger, and the little finger are caught on the edge parts 52A and 52B of the holding surface 52, making it easier to grip the operation part 12 and stabilizing the gripping state.

In addition, the inclined surfaces 58 and 60, which are end parts of the holding surfaces 50 and 52 on the distal end part 22A side, are located on the base end part 26A side with respect to the rotation shafts 31 and 33. Therefore, for example, in a case where the grip part 26 is gripped with the left hand 100, the middle finger 106 can be guided to an optimum position with respect to the inclined surface 60.

In addition, since the end parts of the holding surfaces 50 and 52 on the base end part 26A side have D-cut flat surfaces 62 and 64, respectively, the operator can grip the grip part 26 regardless of a size of the operator's hand.

In addition, since the locking part 40 is provided on the distal end part 22A side with respect to the rotation shafts 31 and 33 of the operation levers 30 and 32, the ultrasound probe 10 can be stably operated by the operation levers.

That is, in a case where the operation part 12 is gripped with the left hand 100 and the operation lever 32 is rotated to be pushed forward (toward the insertion part 14) with the thumb 102, the insertion part 14 (the entire ultrasound probe 10; the same applies below) also tends to move forward, but the index finger 104 hooked on the locking part 40 can absorb a force that causes the insertion part 14 to move forward. That is, the insertion part 14 that tends to move forward can be held by the index finger 104 hooked on the locking part 40 so as not to move forward, thereby rotating the operation lever 32 without moving the insertion part 14 forward.

In a case where a rotation operation of pulling the operation lever 32 backward (toward the cable 28) with the thumb 102 is performed contrary to the above-described rotation operation, the insertion part 14 also tends to move backward, but the index finger 104 hooked on the locking part 40 can absorb a force that causes the insertion part 14 to move backward. That is, the insertion part 14 that tends to move backward can be held by the index finger 104 hooked on the locking part 40 so as not to move backward, thereby rotating the operation lever 32 without moving the insertion part 14 backward.

Therefore, with the operation part 12 of the embodiment, it is possible to stably operate the ultrasound probe 10 with the operation lever.

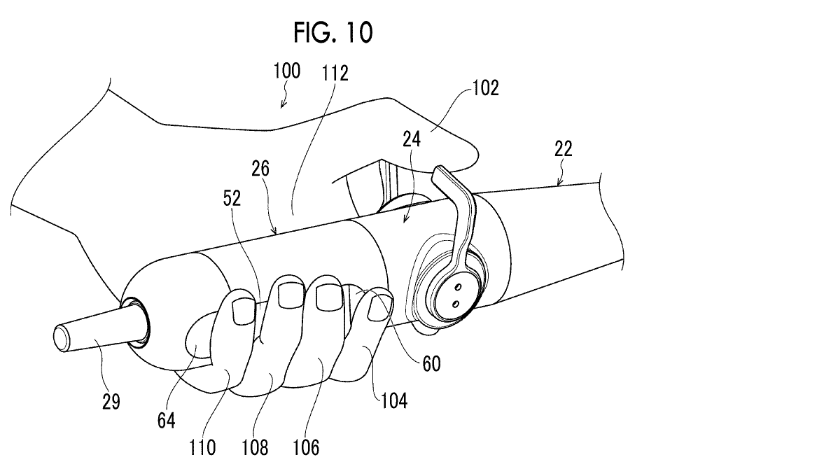

Meanwhile, FIG. 10 is an explanatory view showing a state in which the grip part 26 is gripped and the insertion part 14 is inserted into the body cavity. In this case, since the holding surfaces 50 and 52 of the grip part 26 have the inclined surfaces 58 and 60 on the distal end side, for example, in a case where the grip part 26 is gripped with a palm 112 of the left hand 100, the insertion part 14 can be inserted in a state where the fingertip of the index finger 104 is pressed against the inclined surface 60 and the fingertips of the middle finger 106, a ring finger 108, and a little finger 110 are pressed against the holding surface 52. This allows an insertion force applied by the operator to be effectively transmitted to the insertion part 14.

Hereinafter, some modification examples will be described.

### First Modification Example

In the embodiment, the operation part 12 having the locking part 40 provided in the operation part main body 24 has been described as an example, but, for example, in a case where a length of the operation part main body 24 in the longitudinal axis B direction is short, the locking part 40 may be provided at the operation part-distal end part 22. That is, the locking part 40 in the operation part 12 need only be provided on the distal end part 22A side with respect to the rotation shafts 31 and 33 of the operation levers 30 and 32.

### Second Modification Example

In the embodiment, the operation part 12 in which the holding surfaces 50 and 52 are flat surfaces has been described as an example, but, for example, the holding surfaces 50 and 52 may have a concave surface shape. In this case, since the palm 112 of the left hand 100 fits into the concave surface-shaped holding surface 50 and the fingertips of the middle finger 106, the ring finger 108, and the little finger 110 fit into the concave surface-shaped holding surface 52, the operation part 12 can be stably held.

### Third Modification Example

In the embodiment, the locking part 40 having the recessed groove part 42 has been described as an example, but, for example, the locking part 40 may be a rough surface part that generates frictional resistance, an elastic part that is elastically deformed, or a protruding rib part. In addition, the locking part 40 need only be configured with at least one of the recessed groove part 42, the rough surface part, the elastic part, or the protruding rib part, and, for example, the bottom surface 42A of the recessed groove part 42 may be configured as the rough surface part, a surface of the elastic part may be configured as the rough surface part, or a surface of the protruding rib part may be configured as the rough surface part.

### Fourth Modification Example

In the embodiment, the locking part 40 having the recessed groove part 42 has been described as an example of the indicator part, but, for example, the indicator part need only be a portion that receives the operator’s finger (index finger) and that serves as a mark for identifying where the fingertip of the index finger is to be placed. For example, the indicator part may be formed by marking (painting) the arc surface 24A of the operation part main body 24. By placing the fingertip of the index finger on the mark, the operation lever is easily gripped and easily operated. In addition, by roughening a surface of the mark, a friction coefficient of the surface can be made larger than a friction coefficient of the arc surface 56.

### Other Effects

The holding surfaces 50 and 52 being flat have the following secondary effects. That is, positions where the flat surfaces are formed are aligned with the left-right direction of the bendable part 18. In particular, in a case of an abdominal ultrasound probe, in a case where a grip part is gripped, finger joints are caught on an edge of the grip part. Accordingly, there is an advantage in that, in a case where the bendable part is bent by the operation lever during an examination, the operator can intuitively perceive the bending direction of the bendable part.

In the embodiment, the ultrasound probe 10 has been described as an example of an application of the operation part for a medical device of the embodiment of the present invention, but the present invention can be applied to various operation parts for a medical device without being limited to the ultrasound probe 10. That is, the present invention can be applied to an operation part for a medical device having a bendable insertion part, for example, a laparoscope and an endoscope comprising a soft insertion part. In this case, an observation window is provided on the distal end side of the insertion part.

Although an operation part for a medical device according to the embodiment of the present invention has been described above, the present invention may be improved or modified in some ways without departing from the gist of the present invention.

### Explanation of References

10: ultrasound probe

12: operation part

13: exterior member

14: insertion part

16: insertion part main body

18: bendable part

20: probe

20A: ultrasonic wave emission surface

22: operation part-distal end part

22A: distal end part

24: operation part main body

24A: arc surface

26: grip part

26A: base end part

28: cable

29: bending prevention part

30: operation lever

30A: arm part

30B: finger rest part

30C: rotating portion

30D: circular protruding portion

31: rotation shaft

32: operation lever

32A: arm part

32B: finger rest part

32C: rotating portion

32D: circular protruding portion

33: rotation shaft

40: locking part

42: recessed groove part

42A: bottom surface

42B: distal end-side wall surface

42C: base end-side wall surface

42D: distal end edge part

42E: base end edge part

50: holding surface

50A: edge part

50B: edge part

52: holding surface

52A: edge part

52B: edge part

54: arc surface

56: arc surface

58: inclined surface

60: inclined surface

62: flat surface

64: flat surface

100: left hand

102: thumb

104: index finger

106: middle finger

108: ring finger

110: little finger

112: palm

What is claimed is:

1. An operation part for a medical device, the operation part comprising:
an exterior member that has a distal end part and a base end part and that has a longitudinal axis defined by the distal end part and the base end part;
at least one operation lever that is provided in a first region, which is a partial region, between the distal end part and the base end part of the exterior member and that is configured to be rotatable about a rotation shaft in a state of being erected on one side in a first direction orthogonal to the longitudinal axis;
a first outer peripheral surface that is provided in a second region of the exterior member on a base end part side with respect to the first region and that has a normal component directed toward the other side opposite to the one side in the first direction;
a second outer peripheral surface that is provided in the first region, that has a normal component directed toward the other side in the first direction, and that has a distance to the longitudinal axis equal to or less than a distance from the first outer peripheral surface to the longitudinal axis; and
an indicator part that is provided on a distal end part side with respect to the rotation shaft and on the other side in the first direction.

2. The operation part for a medical device according to claim 1,
wherein an end part of the indicator part on the base end part side is located on the distal end part side with respect to the rotation shaft.

3. The operation part for a medical device according to claim 1, further comprising:
a third outer peripheral surface that is provided in the second region, that is a surface intersecting a second direction orthogonal to each of the longitudinal axis and the first direction, and that has a distance to the longitudinal axis shorter than the distance from the first outer peripheral surface to the longitudinal axis.

4. The operation part for a medical device according to claim 3,
wherein an end part of the third outer peripheral surface on the distal end part side is located on the base end part side with respect to the rotation shaft.

5. The operation part for a medical device according to claim 4,
wherein the third outer peripheral surface is a flat surface.

6. The operation part for a medical device according to claim 1,
wherein the at least one operation lever includes
a first operation lever that bends an insertion part connected to the distal end part in a first bending direction, and
a second operation lever that bends the insertion part in a second bending direction different from the first bending direction, and
a rotation shaft of the first operation lever is formed to extend in a direction in which a rotation shaft of the second operation lever extends.

7. The operation part for a medical device according to claim 1,
wherein the indicator part has a locking surface configured to be hooked by a finger, and
a distance from the locking surface to the longitudinal axis is shorter than the distance from the first outer peripheral surface to the longitudinal axis.

8. The operation part for a medical device according to claim 1,
wherein the indicator part has a friction coefficient greater than a friction coefficient of the first outer peripheral surface.

9. An ultrasound probe comprising:
the operation part for a medical device according to claim 1; and
an insertion part connected to the distal end part of the operation part for a medical device,
wherein a probe is provided on a distal end side of the insertion part.

10. An endoscope comprising:
the operation part for a medical device according to claim 1; and
an insertion part connected to the distal end part of the operation part for a medical device,
wherein an observation window is provided on a distal end side of the insertion part.
